(19) United States
(12) Patent Application Publication
(10) Pub. No.: US 2024/0238353 A1
(43) Pub. Date: Jul. 18, 2024

(54) PROBIOTIC COMPOSITIONS AND USES THEREOF

(71) Applicant: SUPERBREWED FOOD, INC., New Castle, DE (US)

(72) Inventors: Racheli TAMIR, Sugar Land, TX (US); Alon KARPOL, Tel Mond (IL); Aharon EYAL, Jerusalem (IL); Bryan P. TRACY, Wilmington, DE (US)

(21) Appl. No.: 18/286,133

(22) PCT Filed: Apr. 6, 2022
(86) PCT No.: PCT/IB2022/053192

§ 371 Date: Oct. 8, 2023

Related U.S. Application Data
(60) Provisional application No. 63/172,735, filed on Apr. 9, 2021.

(51) Int. Cl. A61K 35/742; A61K 9/00; A61K 9/20; A61K 45/06; A61P 1/14

(52) U.S. Cl. CPC A61K 35/742; A61K 9/0053; A61K 9/20; A61K 45/06; A61P 1/14

(57) ABSTRACT

Provided are methods, compositions, food and beverages, food and beverage additives and kits comprising probiotic microorganisms and uses thereof for treating or preventing a condition caused by eating a cruciferous vegetable or legume in a subject in need thereof. Further provided are compositions comprising an effective dose of an organism within the order Clostridiales and a carrier, wherein said microorganism is present at a concentration of from about 1 to about 10% wt of the total composition.

### CROSS-REFERENCE TO RELATED APPLICATION

The present application gains priority from U.S. Provisional Application No. 63/172,735 filed Apr. 9, 2021 which is incorporated by reference as if fully set-forth herein.

### FIELD OF THE INVENTION

The present invention, in at least some embodiments, relates to probiotic compositions, such as compositions comprising Clostridiales and to uses thereof in treating or preventing a condition caused by eating cruciferous vegetable or legumes.

### BACKGROUND OF THE INVENTION

Cruciferous vegetables are known to be low in calories and to contain a variety of beneficial nutrients, such as folate; vitamins C, E and K; fiber; phytonutrients; and glucosinolates.

Legumes are rich in plant protein, fiber. B-vitamins, iron, folate, calcium, potassium, phosphorus, and zinc.

However, consumption of cruciferous vegetables and beans or legumes is known to cause various side-effects. The most common side effects are those which affect the digestive system, such as stomach rumbling, bloating and constipation.

### SUMMARY OF THE INVENTION

According to an aspect of some embodiments of the present invention, there is provided a method of treating or preventing a condition caused by eating cruciferous vegetables or legumes, the method comprising administering to a subject in need thereof at least one probiotic microorganism.

According to a further aspect of some embodiments of the present invention, there is provided a composition comprising at least one probiotic microorganism, for use in treating a condition caused by eating cruciferous vegetables or legumes.

According to a further aspect of some embodiments of the present invention, there is provided a composition comprising an effective dose of an organism within the order Clostridiales and a carrier, wherein said microorganism is present at a concentration of from about 1 to about 10% wt of the total composition.

Further provided are foods, beverages, food additives and kits comprising a composition as disclosed herein.

### DETAILED DESCRIPTION OF THE INVENTION

The present invention relates to methods and compositions comprising probiotic microorganisms, such as Clostridiales and uses thereof in treatment of conditions caused by eating cruciferous vegetables or legumes.

The present inventors have surprisingly found that at least one undesirable side effect in a subject caused by eating a cruciferous vegetable or legume can be partially or fully treated or prevented by administering to the subject a probiotic microorganism, such as Clostridiales.

The particulars shown herein are by way of example and for purposes of illustrative discussion of the various embodiments of the present invention only and are presented in the cause of providing what is believed to be the most useful and readily understood description of the principles and conceptual aspects of the invention. In this regard, no attempt is made to show details of the invention in more detail than is necessary for a fundamental understanding of the invention, the description making apparent to those skilled in the art how the several forms of the invention may be embodied in practice.

The present invention will now be described by reference to more detailed embodiments. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention.

As used herein, the term “treating” includes preventing, curing, ameliorating, mitigating, and reducing the instances or severity of a condition or a symptom thereof.

As used herein, the term “administering” includes any mode of administration, such as oral, subcutaneous, sublingual, transmucosal, parenteral, intravenous, intra-arterial, buccal, sublingual, topical, vaginal, rectal, ophthalmic, otic, nasal, inhaled, intramuscular, intraosseous, intrathecal, and transdermal, or combinations thereof. “Administering” can also include providing a different compound that when ingested or delivered as above will necessarily transform into the compound that is desired to be administered, this type of “different compound” is often being referred to as a “Prodrug”. “Administering” can also include prescribing or filling a prescription for a dosage form comprising a particular compound. “Administering” can also include providing directions to carry out a method involving a particular compound or a dosage form comprising the compound or compounds.

As used herein, the term “effective dose” means the amount of a probiotic microorganism that, when administered to a subject for treating a disease, disorder, or other undesirable medical condition, is sufficient to have a beneficial effect with respect to that disease, disorder, or condition. The effective dose will vary depending on the identity and formulation form of the active substance, the disease or condition and its severity, and the age, weight, and other relevant characteristics of the patient to be treated. Determining the effective amount of a given active substance is within the ordinary skill of the art and typically requires no more than routine experimentation.

As used herein, the term “probiotic microorganism” refers to a live microorganism which provides health benefits to an animal when consumed, generally by restoring the balance of gut flora.

As used herein, the term “acetogen” refers to an obligately anaerobic bacterium that uses the reductive acetyl-CoA or Wood-Ljungdahl pathway as its main mechanism for energy conservation and for synthesis of acetyl-CoA and cell carbon from CO₂.

As used herein, the term “spore former” refers to a microorganism capable of forming spores, which are highly resistant, dormant structures having no metabolic activity, formed in some microorganisms in response to environmental conditions (such as depletion of nutrients) in order to help in survival of the microorganism.

As used herein, the term “vegetative cell” refers to a bacterial cell that is actively growing rather than forming spores.

As used herein, the term “prebiotic” refers to a compound that induces the growth or activity of a probiotic.

As used herein, the term “pharmaceutically acceptable carrier” means a substance or collection of substances capable of being combined with an active ingredient that is suitable for administration to an animal. The carrier may comprise a liquid or solid filler, diluent, excipient, solvent or encapsulating material.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

As used herein, when a numerical value is preceded by the term “about”, the term “about” is intended to indicate +/−10% of that value.

As used herein, the terms “comprising”, “including”, “having” and grammatical variants thereof are to be taken as specifying the stated features, integers, steps or components but do not preclude the addition of one or more additional features, integers, steps, components or groups thereof. These terms encompass the terms “consisting of” and “consisting essentially of”.

Additional advantages of the invention will be set forth in part in the description which follows, and in part will be obvious from the description, or may be learned by practice of the invention. It is to be understood that both the foregoing general description and the following detailed description are exemplary and explanatory only and are not restrictive of the invention, as claimed.

According to an aspect of some embodiments of the present invention, there is provided a method of treating a condition caused by eating cruciferous vegetables or legumes, the method comprising administering to a subject in need thereof at least one probiotic microorganism.

According to a further aspect of some embodiments of the present invention, there is provided a composition comprising at least one probiotic microorganism, for use in treating a condition caused by eating cruciferous vegetables or legumes.

According to some embodiments, said condition is selected from the group consisting of borborygmus (stomach rumbling), bloating, constipation and combinations thereof.

According to some embodiments, said microorganism is of the class clostridia.

According to some embodiments, said microorganism is of an order selected from the group consisting of Clostridiales, Halanaerobiales, Natranaerobiales, Syntrophomonadaceae, Lachnospiraceae, Ruminococcaceae and combinations thereof. According to some such embodiments, said microorganism is of a genus selected from the group consisting of autoethanogenum, carboxidivorans, coskatii, drakei, kluyveri, ljungdahlii, magnum, ragsdalei, scatologenes, tyrobutyricum, haemolyticum, novyi, collagenovorans, estertheticum, proteolyticum, senegalense, sulfidigenes, tepidiprofundi, tunisiense, paraputrificum, baratii, colicanis, chauvoei, disporicum, sariagoforme, saudiense, septicum, fallax, (Sarcina) ventriculi, algidicarnis, cadaveris, hydrogeniformans and combinations thereof.

According to some embodiments, said at least one probiotic microorganism comprises two or more different types of microorganisms (such as two, three, four, five or more different types of microorganisms).

According to some embodiments, each type of microorganism is selected from a different order. According to some embodiments, each type of microorganism is selected from the same order.

According to some embodiments, each type of microorganism is selected from a different genus. According to some embodiments, each type of microorganism is selected from the same genus.

According to some embodiments, each type of microorganism is selected from a different group. According to some embodiments, each type of microorganism is selected from the same group.

According to some embodiments, said organism is an acetogen.

According to some embodiments, said microorganism is configured to hydrolyze at least one selected from the group consisting of raffinose; oligo-, di-, or mono-saccharides; glucans; and polyols.

According to some embodiments, said organism is a spore former.

According to some embodiments, said organism is a vegetative cell.

According to some embodiments, said organism comprises both spores and vegetative cells.

According to some embodiments, said treating comprises oral administration.

According to some embodiments, said microorganism is administered in the form of a pill, tablet, capsule, syrup, solution, suspension, powder or the like, or any combination thereof. According to one such embodiment, said microorganism is administered in the form of a capsule. According to one such embodiment, said microorganism is administered in the form of a tablet.

According to some embodiments, said microorganism is provided in a dosage form having a weight of from about 0.1 to about 0.1 gram, wherein said dosage form comprises at least 104 colony forming units (CFU) of said microorganism/gram.

According to some embodiments, said administration further comprises administering a pharmaceutically acceptable carrier and a prebiotic, such as a fibre selected from the group consisting of fructooligosaccharides (FOS), inulins, galactooligosaccharides (GOS), resistant starch, pectin, beta-glucans, xylooligosaccharides, mannan oligosaccharide and combinations thereof.

According to some embodiments, said prebiotic is administered together with the microorganism in a single dosage form.

According to some embodiments, said microorganism and said prebiotic are administered in separate dosage forms. According to some such embodiments, said microorganism and said prebiotic are administered independently, sequentially (wherein said microorganism is administered prior to administration of said prebiotic, or wherein said prebiotic is administered prior to administration of said microorganism), simultaneously or concomitantly.

According to some embodiments, wherein said microorganism and said prebiotic are administered in separate dosage forms, the separate dosage forms may be separate dosage forms of the same type e.g. each of said microorganism and said prebiotic are administered separately as a capsule. Alternatively, the separate dosage forms may be separate dosage forms of different types e.g. said microorganism may be administered as an emulsion and said prebiotic may be administered as a pill.

According to some embodiments, said administration is carried out multiple times per day, such as twice, three times, four times or six times per day.

According to some embodiments, said administration is carried out for a period of one days, two days, three days, four days, five days, six days, one week, two weeks, three weeks, or one month.

According to some embodiments, said administration is daily administration.

According to some embodiments, said microorganism is administered as required, prior to, together with or after eating said cruciferous food or legume.

According to some embodiments, said subject is a human subject.

According to some embodiments, said subject is a non-human animal. According to some such embodiments, said non-human animal is selected from the group consisting of a domestic animal (such as a cat, dog, hamster, gerbil, mouse, rabbit or the like) and a farm animal (such as a horse, cow, sheep, goat, duck, chicken and the like).

According to an aspect of some embodiments of the present invention, there is provided a composition comprising an effective dose of an organism within the order Clostridiales and a carrier, wherein said microorganism is present at a concentration of from about 1 to about 10% wt of the total composition, such as about 1% wt, about 2% wt, about 3% wt, about 4% wt, about 4% wt, about 6% wt, about 7% wt, about 8% wt, about 9% wt or about 10% wt.

According to some embodiments, said microorganism is of an order selected from the group consisting of Clostridiales, Halanaerobiales, Natranaerobiales, Syntrophomonadaceae, Lachnospiraceae, Ruminococcaceae and combinations thereof.

According to some embodiments, said microorganism is of a genus selected from the group consisting of autoethanogenum, carboxidivorans, coskatii, drakei, kluyveri, ljungdahlii, magnum, ragsdalei, scatologenes, tyrobutyricum, haemolyticum, novyi, collagenovorans, estertheticum, proteolyticum, senegalense, sulfidigenes, tepidiprofundi, tunisiense, paraputrificuni, baratii, colicanis, chauvoei, disporicum, sartagoforme, saudiense, septicum, fallax, (Sarcina) ventricoli, algidicarnis, cadaveris, hydrogeniformans and combinations thereof.

According to some embodiments, said at least one probiotic microorganism comprises two or more different types of microorganisms (such as two, three, four, five or more different types of microorganisms).

According to some embodiments, each type of microorganism is selected from a different order. According to some embodiments, each type of microorganism is selected from the same order.

According to some embodiments, each type of microorganism is selected from a different genus. According to some embodiments, each type of microorganism is selected from the same genus.

According to some embodiments, each type of microorganism is selected from a different group. According to some embodiments, each type of microorganism is selected from the same group.

According to some embodiments, said organism is an acetogen.

According to some embodiments, said microorganism is configured to hydrolyze at least one selected from the group consisting of raffinose; oligo-, di-, or mono-saccharides; glucans; and polyols.

According to some embodiments, said organism is a spore former.

According to some embodiments, said organism is a vegetative cell.

According to some embodiments, said organism comprises both spores and vegetative cells.

According to some embodiments, said composition is in the form of a pill, tablet, capsule, syrup, solution, suspension, powder or the like, or any combination thereof. According to one such embodiment, said composition in the form of a capsule. According to one such embodiment, said composition in the form of a tablet.

According to some embodiments, said microorganism is provided in a dosage form having a weight of from about 0.1 to about 0.1 gram, wherein said dosage form comprises at least 104 colony forming units (CFU) of said microorganism/gram.

According to some embodiments, said composition further comprises at least one selected from the group consisting of soluble fibers, water, phosphate, short fatty acids and combinations thereof.

According to some embodiments, said short fatty acids are selected from the group consisting of acetic acid, propionic acid, butyric acid, valeric acid, formic acid, isovalerate, 2-methylbutanoic acid and combinations thereof.

According to some embodiments, short fatty acids are derived from at least one selected from the group consisting of pectin, starch, or an extract of oatmeal, brown rice, nuts (such as almonds), fruits (such as apples, strawberries, avocados) or vegetables (such as carrots).

According to some embodiments, said composition further comprises a prebiotic, such as a fibre selected from the group consisting of fructooligosaccharides (FOS), inulins, galactooligosaccharides (GOS), resistant starch, pectin, beta-glucans, xylooligosaccharides, mannan oligosaccharide and combinations thereof.

According to an aspect of some embodiments of the present invention, there is provided a food or beverage comprising the composition as disclosed herein.

According to some embodiments, said food is a dairy product, such as one selected from the group consisting of yogurt, milk and cheese.

According to some embodiments, said food or beverage further comprises an extract of a fruit or vegetable, such as one selected from the group consisting of banana, papaya, pineapple, asparagus, cucumber and combinations thereof.

According to some embodiments, said beverage is selected from the group consisting of tea, coffee, cocoa, a soft drink and an energy drink (i.e. a drink comprising a stimulant such as caffeine). According to some embodiments, an energy drink comprises caffeine at a concentration of about 30-40 mg/ml and optionally further comprises at least one selected from the group consisting of sugar, amino acids (such as taurine), electolytes, vitamins and herbal extracts.

According to some embodiments, there is provided a food or beverage additive comprising the composition as disclosed herein, for adding to a food or beverage prior to consumption.

According to some embodiments, there is provided a kit comprising the composition as disclosed herein and a prebiotic.

According to some embodiments, the composition as disclosed herein is for use in treating a condition caused by eating a cruciferous vegetable or a legume. According to some such embodiments, the condition is selected from the group consisting of borborygmus (stomach rumbling), bloating, constipation and combinations thereof.

According to some embodiments of the method, composition, food, food additive or kit disclosed herein, the cruciferous vegetable is any vegetable of the Brassicaceae family, such as one selected from the group consisting of broccoli, cabbage, brussels sprouts, cauliflower, horseradish, cress, kale, collard greens, bok choy, turnip roots, swede, mustard seeds, argula, radish, kohlrabi, and the like.

According to some embodiments of the method, composition, food, food additive or kit disclosed herein, the legume is any plant in the family Fabaceae or the fruit or seed of such a plant, such as a dry grain of the seed (commonly referred to as a pulse). Non-limiting examples of legumes include beans (such as kidney beans, lima beans, adzuki beans, mung beans, runner beans, broad beans), garden peas, chickpeas and lentils (such as red, green or brown lentils).

### Example

A clinical trial is conducted on a group of 200 human subjects (100 males and 100 females), between the ages of 15 and 75 to measure the use of probiotics to mitigate the symptoms associated with Raffinose intolerance. A placebo probiotic in the form of a pill comprising only excipient, is administered to half of the total number of subjects (the control group). A probiotic pill comprising 10⁹ CFU of an acetogen from the clostridia class that is able to hydrolyze or consume raffinose is administered to each of the remaining subjects (the test group).

Twenty four hours after receiving the placebo pill or the probiotic pill, both groups are provided once a day a meal including 100 grams of baked beans. During the course of the study (28 days), each of the subjects in both groups receive 100 grams of baked beans each day. Each member of the control group receives a placebo pill each day, while each member of the test group receives 10⁹ CFU of an acetogen from the clostridia class that is able to hydrolyze or consume raffinose each day.

Throughout the 28 day study period, each subject submits a report each day regarding the following side effects: bloating, diarrhea, nausea, indigestion, vomiting, loss of appetite, and stomach cramping or pain.

In the control group, bloating, diarrhea, nausea, indigestion, vomiting, and stomach cramping are reported for 20, 10, 5, 1, 4, 4, 25 patients respectively. In the test group, bloating, diarrhea, nausea, indigestion, vomiting, and stomach cramping are reported for 5, 2, 1, 0, 0, 1, 8 patients respectively

Overall, it is observed that administration of probiotics at 10⁹ CFU of an acetogen strain from the clostridia class that is able to hydrolyze or consume raffinose, for 28 days reduces symptoms associated with raffinose intolerance.

1-45. (canceled)

46. A method of treating a condition caused by eating cruciferous vegetables or legumes, the method comprising administering to a subject in need thereof at least one microorganism of the class clostridia.

47. The method of claim 46, wherein said condition is selected from the group consisting of borborygmus (stomach rumbling), bloating, constipation and combinations thereof.

48. The method of claim 47, wherein said microorganism is of an order selected from the group consisting of Clostridiales, Halanaerobiales, Natranaerobiales, Thermoanaerobacterales, Lachnospiraceae, Ruminococcaceae and combinations thereof.

49. The method of claim 47, wherein said microorganism is of a genus selected from the group consisting of as Acetivibrio, Acidaminobacter, Alkaliphilus, Anaerobacter, Caloramator, Magnum, Caloranaerobacter, Coprobacillus, Dorea, Natronincola, Oxobacter, Sarcina, Sporobacter, Thermobrachium, Thermohalobacter, Tyrobutyricum Tindallia, autoethanogenum, carboxidivorans, coskatii, drakei, ljungdahlii, ragsdalei, sulfidigenes, tepidiprofundi, tunisiense, saudiense, hydrogeniformans, Blautia, Eubacterium and combinations thereof.

50. The method of claim 46, wherein said organism is an acetogen.

51. The method of claim 46, wherein the microorganism is configured to hydrolyze at least one selected from the group consisting of raffinose; oligo-, di-, or mono-saccharides; glucans; and polyols.

52. The method of claim 46, wherein said organism is selected from the group consisting of a spore former, a vegetative cell and a combination thereof.

53. The method of claim 46, further comprising administering a pharmaceutically acceptable carrier and a prebiotic.

54. The method of claim 46, wherein said microorganism is administered in a form selected from the group consisting of a capsule and a tablet.

55. The method of claim 46, wherein said administering comprises oral administration.

56. The method of claim 46, wherein said administering is daily administration.

57. The method of claim 46, wherein said subject is a human or non-human animal, optionally wherein said non-human animal is selected from the group consisting of a domestic animal and a farm animal.

58. A composition comprising an effective dose of an organism within the class Clostridia and a carrier, wherein said microorganism is present at a concentration of from about 1 to about 10% wt of the total composition.

59. The composition of claim 58, wherein said microorganism is of an order selected from the group consisting of Clostridiales, Halanaerobiales, Natranaerobiales, Syntrophomonadaceae, Lachnospiraceae, Ruminococcaceae and combinations thereof.

60. The composition of claim 59, wherein said microorganism is of a genus selected from the group consisting of as Acetivibrio, Acidaminobacter, Alkaliphilus, Anaerobacter, Caloramator, Magnum Caloranaerobacter, Coprobacillus, Dorea, Natronincola, Oxobacter, Sarcina, Sporobacter, Thermobrachium, Thermohalobacter, Tyrobutyricum Tindallia, autoethanogenum, carboxidivorans, coskatii, drakei, ljungdahlii, ragsdalei, sulfidigenes, tepidiprofundi, tunisiense, saudiense, hydrogeniformans and combinations thereof.

61. The composition of claim 58, wherein said organism is an acetogen.

62. The composition of claim 58, where the microorganism is configured to hydrolyze at least one selected from the group consisting of raffinose; oligo-, di-, or mono-saccharides; glucans; and polyols.

63. The composition of claim 58, wherein said organism is selected from the group consisting of a spore former, a vegetative cell and a combination thereof.

64. The composition of claim 58, provided in a form selected from the group consisting of a capsule and a tablet.

65. The composition of claim 58, provided in the form of a food or beverage selected from the group consisting of chocolate, a shake, yoghurt, milk, fruit juice, bread, oatmeal, a protein bar, a cake or a cupcake.
